(19) United States
(12) Patent Application Publication
(10) Pub. No.: US 2007/0280850 A1
(43) Pub. Date: Dec. 6, 2007

(54) Mri Compatible Devices

(76) Inventor: James M. Carlson, Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/662,126

(22) PCT Filed: Sep. 23, 2005
(86) PCT No.: PCT/US05/34143

§ 371 Date: Jul. 24, 2007

Related U.S. Application Data
(60) Provisional application No. 60/613,393, filed on Sep. 27, 2004.

(51) Int. Cl. A61L 31/18; A61F 2/06; A61F 2/52; A61L 31/02

(52) U.S. Cl. 420/580; 604/6.06; 623/1.13; 623/1.15

(57) ABSTRACT

A medical device suitable for use with magnetic resonance imaging techniques includes a component that is formed from a refractory metal, a precious metal, an alloy comprising a refractory metal, an alloy comprising a precious metal, and/or alloy comprising at least one refractory metal and at least one precious metal. The component has a magnetic susceptibility less than about 300×10⁻⁶ cgs, and has a low radiopacity such that the component can be visualized under fluoroscopy.

### RELATED APPLICATION

The present patent document claims the benefit of the filing date under 35 U.S.C. §119(e) of Provisional U.S. Patent Application Ser. No. 60/613,393, filed Sep. 27, 2004, which is hereby incorporated by reference.

### BACKGROUND

1. Technical Field

The present application relates generally to medical devices. More particularly, the application relates to devices for medical diagnostic and/or interventional use that are compatible with medical resonance imaging equipment, and that exhibit sufficient radiopacity to be observable during x-ray fluoroscopy.

2. Background Information

Magnetic resonance imaging, commonly known as MR imaging or simply MRI, is a technique that has gained increased importance in the medical field in recent years. This procedure is driven by a complex interaction of magnetic and radio frequency fields. MRI is useful for providing non-invasive medical diagnostic information in real time, and also as an imaging tool for use during various interventional procedures. Unlike many other imaging techniques, MRI provides excellent soft tissue contrast, and provides the ability to visualize images in very high resolution. This technique is often favored over other imaging techniques because it does not require the use of ionizing radiation and toxic iodinated contrast agents, and its use does not require that technicians and patients be outfitted with leaded or other protective gear.

One difficulty that has been encountered with MRI procedures is that it is often difficult to adequately distinguish and identify certain interventional medical devices, such as catheters, wire guides and the like, on the resulting image. Some of these devices, particularly those formed from certain polymers, are virtually invisible to MR imaging. As a result, the physician cannot adequately determine the position of the device. Other devices, such as those formed from austenitic stainless steel, or from some nickel or cobalt-based alloys, may cause an artifact to appear on the medical image in place of the device. In medical terminology, an “artifact” is an artificial or distorted feature which appears in an image, and which is not present in the original imaged object. In MRI applications, an artifact may comprise a white area or “void” that is present on an image that obscures the actual device or tissue segment of interest in the examination. On other occasions, an artifact may be present as a blurred or cloudy portion of the image. Artifacts not only obscure adjacent tissue in the image, but also bodily fluids, such as blood, bile, urine, etc. Depending on the size of the artifact and the type of procedure, the presence of the artifact may make it virtually impossible to carry out certain procedures under magnetic resonance imaging. In an image in which the physician may be searching for minute signals or indications, the presence of an artifact can virtually destroy the viability of the MRI procedure.

Artifacts are created when medical devices are manufactured using materials that have high magnetic susceptibility. The magnetic susceptibility of a material is the ability of the material to become magnetized by an externally applied magnetic field. Commonly used magnetic metals, such as iron, have a high magnetic susceptibility. Certain alloys commonly used in medical devices also have particularly high magnetic susceptibility. As a result, the presence of these metals or metallic alloys in a medical device creates significant artifact during MR imaging. Although the use of such alloys may be desirable in some medical applications, their high magnetic susceptibility makes them less than desirable for medical resonance imaging. Magnetic susceptibility is generally considered to be proportional to artifact size. In general, the higher the magnetic susceptibility of a material, the more artifact is created and the less suitable the material is for use in MRI. On the other hand, a material having low magnetic susceptibility is more likely to be suitable for MRI.

Medical devices that incorporate metal or metal alloys having high magnetic susceptibility, such as braided and coiled catheters and wire guides, can therefore create a large artifact when inserted in MRI equipment. Depending upon the size of the artifact or void, and the anatomy or type of procedure to be performed, this artifact may cause some procedures to be virtually impossible to carry out under magnetic resonance imaging. Many newer alloys used in medical procedures, such as L-605, MP35N, nitinol, Elgiloy, Inconel alloy 625 and various other nickel and cobalt-based superalloys, have a lower magnetic susceptibility than older materials such as 304 or 316 stainless steel. Medical devices that incorporate these metals or metal alloys generally have reduced artifact when compared to the use of more traditional materials. However, such devices may still create a higher than desired level of artifact when subjected to a magnetic field. Some of the newer alloys also suffer from low radiopacity, making them difficult to properly identify under x-ray fluoroscopy. Thus, when these materials are used in a medical device such as a braid-reinforced catheter or a coil-reinforced catheter, the device requires the incorporation of a radiopaque filler, such as tungsten, BiOCl, barium sulfate, or the like, in order to be observable during fluoroscopic evaluation.

Accordingly, it would be desirable to provide medical interventional devices that are compatible with magnetic resonance imaging procedures, and that have sufficient radiopacity such that it is not necessary to incorporate a radiopaque filler into the polymer. Such devices would provide satisfactory MR imaging, would be observable under fluoroscopy, and would exhibit satisfactory biocompatibility and mechanical properties.

### BRIEF SUMMARY

The problems of the prior art are addressed by the present invention. In one embodiment, the invention comprises an MRI compatible medical device comprising a wire or other structure formed from a metal or a metal alloy. The metal or metal alloy comprises a member selected from the group consisting of refractory metals, precious metals, refractory metal alloys, precious metal alloys, and mixtures of the foregoing. The metal or metal alloy has a magnetic susceptibility less than about 300×10⁻⁶ cgs, and has a radiopacity sufficient for visualization under fluoroscopy. In a preferred embodiment, the refractory metal can comprise one or more of molybdenum, tungsten, tantalum, titanium, niobium and hafnium, and the precious metal can comprise one or more of platinum, rhenium, rhodium, gold, palladium, ruthenium, silver, iridium and osmium. The alloy can comprise two or more of the aforementioned metals alloyed together.

### DETAILED DESCRIPTION OF THE PRESENTLY PREFERRED EMBODIMENTS

The present invention relates generally to devices for medical diagnostic and/or interventional use, and to metal and metal alloy formulations that may be utilized in such devices. The metals and metal alloys have low magnetic susceptibility, to enable the devices to be compatible with medical resonance imaging equipment. In addition, the metals and metal alloys exhibit sufficient radiopacity such that they are observable under x-ray fluoroscopy.

Many well-known metals and alloys, such as stainless steel and various cobalt or nickel-based alloys, have long been utilized in medical devices. These materials have sufficient strength for most intended uses, have sufficient density for visualization under x-ray fluoroscopy, and have sufficient biocompatibility for use in a wide variety of medical applications. Unfortunately, many such materials also have a high magnetic susceptibility. As a result, they create an undesirably large artifact under MRI, and are therefore generally unsuitable for such use. Many newer alloys, such as L-605, MP35N, nitinol, Elgiloy and Inconel alloy 625 have lower magnetic susceptibility when compared to stainless steel and some of the earlier metals and alloys. However, the magnetic susceptibility of these newer materials, and therefore the amount of artifact created, is still greater than desired. Some of the newer alloys also suffer from low radiopacity, making them difficult to properly identify under x-ray fluoroscopy.

A class of materials identified herein for use in medical devices has lower magnetic susceptibility than currently used materials, and as a result, exhibits reduced artifact under MRI when compared to those materials. In addition, the class of materials exhibits favorable radiopacity, thereby permitting the materials to be viewed under x-ray fluoroscopy. The materials also exhibit favorable mechanical properties for use in a medical device, and are generally considered biocompatible for medical use.

Generally speaking, the class of materials utilized in the present invention includes refractory metals and precious metals, as well as refractory metal alloys, precious metal alloys, and alloys containing a refractory metal and a precious metal. Refractory metals and precious metals are known to have the beneficial properties of a very high melting point and low magnetic susceptibility. In addition, suitable alloys can be prepared from these metals to retain, or even improve upon, these beneficial properties.

Suitable refractory metals for use in the inventive devices include molybdenum, tungsten, tantalum, titanium, niobium, and hafnium. Suitable precious metals include platinum, rhenium, rhodium, gold, palladium, ruthenium, silver, iridium and osmium. Alloys of the foregoing metals may be formed by alloying together two or more refractory metals, two or more precious metals, or two or more metals selected from the group consisting of refractory metals and precious metals. As stated, the alloys may be specifically formulated to retain or enhance the desirable physical properties of the pure metals.

The metals and alloys referred to herein will generally comprise at least about 50 weight percent of refractory metals and/or precious metals. Thus, although other materials may be present in the metal or alloyed component described herein, the total amount of refractory metal and/or precious metal will generally be at least about 50 weight percent. For example, an alloy may comprise 40 weight percent of a refractory metal A and 10 weight percent of a refractory metal B. An alloy may also comprise about 40 weight percent of a precious metal A and 10 weight percent of a precious metal B. As another example, an alloy may comprise about 40 weight percent of a refractory metal A and 10 weight percent of a precious metal B.

Although the metals and alloys preferably comprise at least about 50 weight percent of refractory and/or precious metals, it is preferred that such refractory and/or precious metal composition exceed 50 weight percent. Thus, higher amounts, such as 70 weight percent, 90 weight percent, 95 weight percent, and others, are more preferred. The higher weight percent will generally provide a metallic component having sufficient strength for its intended use, provide a component having sufficient magnetic susceptibility for the intended use, be biocompatible, and have sufficient density such that the component is visible under fluoroscopy.

Those skilled in the art will thus appreciate that the specific amounts of the metals described herein can be varied depending upon the intended use of the medical device. Desirable properties such as MRI compatibility, device strength, radiopacity, biocompatibility, and other known mechanical properties may vary in importance in a particular medical application. A skilled artisan can readily formulate a suitable MR compatible metallic composition for a particular use based upon the principles provided herein.

In addition to the pure metals listed above, the following examples comprise a non-limiting list of suitable alloys that may be formulated from these refractory metals and precious metals: (a), molybdenum combined with about 5 to 50 percent rhenium; (b) tantalum combined with about 0 to 10% tungsten and/or about 0 to 3% hafnium; (c) niobium combined with about 0 to 4% zirconium and/or about 0 to 30% tantalum and/or about 0 to 15% tungsten and/or about 0 to 1% titanium; (d) tungsten combined with about 20 to 30% rhenium; (e) platinum combined with about 0 to 30% iridium; (f) palladium combined with about 0 to 30% silver and/or about 0 to 10% copper; (g) palladium combined with about 0 to 20% rhenium; and (h) palladium combined with about 0 to 20% ruthenium. Unless specified otherwise, all percentages referred to herein are weight percent.

Those skilled in the art will appreciate that when the weight percent of a component is provided as a range, that the range includes all amounts between the two end points of the range. Preferably, the base metals utilized in the alloys are provided in a chemically pure state. In the examples, the first-listed metal comprises the majority metal, and the total of the metals will generally comprise about 100%. Due to the possible presence of impurities and minor amounts of known additives, it is not necessary in each case that the total of the metallic component comprise exactly 100%. However, in each case, the combined weight percent of the majority metal and the remaining metal(s) will be at least about 50%.

In addition to the foregoing examples, utilizing modem metallurgical practices such as powder metallurgy and hot isostatic pressing, a combination alloy of a refractory metal/alloy with a precious metal/alloy may be formulated. A non-limiting list of suitable combination alloys includes, e.g., (a) molybdenum combined with 5 to 30% rhenium and 5 to 30% palladium; (b) tantalum with 5 to 40% palladium combined with about 0 to 30% silver and/or about 0 to 10% copper; and (c) niobium combined with 1 to 5% zirconium and/or 5 to 40% palladium.

Those skilled in the art will appreciate that the particular refractory metal and/or precious metal utilized in a particular alloy, and the percentages of the refractory metal(s) and/or the precious metal(s) utilized in a particular alloy according to the present invention may be varied based upon the type of device in which the metal or alloy is to be used, and upon the particular properties of interest in the device. An alloy may be formulated such that the magnetic susceptibility of the alloy is greater than, or less than, the magnetic susceptibility of the base metal. However, in order to achieve the benefits of the present invention, the magnetic susceptibility of the resulting alloy should be sufficient to obtain the desired results, that is, to avoid creating an unreasonable amount of artifact. Preferably, the magnetic susceptibility of the metal or alloy will not be greater than about 300×10⁻⁶ cgs, more preferably not greater than about 200×10⁻⁶ cgs, and most preferably not greater than about 100×10⁻⁶ cgs.

In addition to having low magnetic susceptibility, refractory metals and precious metals, and alloys of them, are favorable for use in the inventive class of materials because they are very dense metals. As a result, these metals and alloys are readily visible under x-ray fluoroscopy. Generally, in order to provide sufficient visibility under fluoroscopy, a metal or alloy should have a density of at least about 4 g/cm³, preferably at least about 8 g/cm³, and more preferably at least about 10 g/cm³.

Visibility under fluoroscopy is a desirable quality in a medical device, because it enables the physician to examine and treat the patient without the necessity of utilizing a MR scanner. Certain medical procedures may be easier to perform while using a fluoroscope due to the confining space within a MR scanner. In addition, use of a fluoroscope is generally less costly than utilizing a MR scanner. On the other hand, MRI provides excellent soft tissue contrast when compared to fluoroscopy, and provides the ability to visualize images in very high resolution. In addition, MR scanners offer the advantage that the doctors and medical assistants need not wear radiation shielding garments. Over time, the use of such garments may cause chromic back discomfort, joint fatigue and other work related problems for the clinician and staff personnel. In certain situations, a patient may be allergic to the contrast agent, making angiography or fluoroscopy impossible and life threatening to perform. Thus, for at least the foregoing reasons, hospitals and other medical facilities often include both fluoroscopy and MR together in a surgical suite. In this way, a specific medical procedure may be optimized by utilizing the best possible technique based upon the unique factors that must be accounted for in the particular procedure at hand.

The alloys described herein can be formulated to exhibit specific properties with a wide range of possible mechanical properties. Mechanical properties that are often significant in medical devices include ultimate tensile strength (UTS, reported in ksi), 0.2% offset yield strength (YS, reported in ksi), elongation (reported in %) and Young's Modulus (E, reported in msi). With the present invention, a medical device can be provided that includes a specific metal or alloy having suitable properties for a particular task.

Thus, for example, in some medical applications it may be desired to utilize a medical device formed from a relatively soft and pliable metal or alloy. One example of such use would be as a wire guide or introducer sheath that must navigate tortuous pathways in the brain or other areas of the body having small arteries or veins. In this case, the wire guide can comprise a palladium—rhenium alloy, such as Pd—10% Re, available from Johnson Matthey North America, of West Chester, Pa. According to convention in the metallurgical field, Pd-10% Re indicates that the composition includes 90% of the dominant metal, in this case palladium, and 10% of a metal present in a lesser amount, in this case rhenium. This alloy has a Young's modulus of 205 ksi and 11 msi, respectively. The Pd-10% Re alloy is highly radiopaque and is MR compatible. Other soft and pliable refractory metals, precious metals, and alloys of the foregoing, such as platinum or platinum alloys, can also be favorably utilized for this purpose. However, due to cost considerations, the use of palladium will generally be preferred over platinum.

On the other hand, in applications such as coronary angioplasty, a stronger, stiffer wire guide material would normally be preferred. Thus, when a cardiologist is conducting a balloon angioplasty or stenting procedure in a coronary artery that is partially blocked by a heavily calcified plaque, a catheter or wire guide is desired that has different properties as compared to a catheter or wire guide required by a neurologist who is treating a patient with a brain aneurism in a small artery of the brain. In the case of an angioplasty procedure, a strong and stiff wire guide manufactured using, e.g., a cold worked 304V stainless steel wire might ordinarily be desirable. 304V stainless steel wire has an ultimate tensile strength (UTS) of 310 ksi and a Young's modulus (E) of 29 msi. However, this wire guide would create significant artifact under MR imaging. A wire guide comprising a cold worked alloy known as Moly-Rhenium (Mo-47.5% Re), available from Rhenium Alloys, Inc. of Elyria, Ohio, could be substituted in this case for the 304V stainless steel in order to produce a highly radiopaque and MR compatible device. The Mo-47.5% Re wire can be work hardened to achieve a satisfactory ultimate tensile strength of 355/566 ksi and Young's modulus of 50.8 msi. Likewise, other refractory metals, precious metals, and alloys of the foregoing, having desirable physical properties for the task at hand may be substituted.

Thus, it will be appreciated that the particular mechanical property desired for a particular medical procedure, whether it be ultimate tensile strength, 0.2% offset yield strength, percent elongation, Young's modulus, or any combination of properties, depends upon the specific application in which the device will be employed. In certain cases, it may be desirable to have high stiffness and ultimate tensile strength, and in other cases flexibility and tortuousity may be more important properties. When utilizing the teachings of the present application, one of ordinary skill in the art can readily provide a suitable MR compatible metal or alloy suitable for a particular medical application without undue experimentation.

Table 1 comprises a non-limiting list of examples of suitable metals and alloys that may be used in the medical devices of the present invention. The metals and alloys listed in Table 1 are commercially available from the sources listed in the Table, among others. The metals and alloys are believed to be particularly suitable for use as braided wire in braid-reinforced sheaths and catheters, coils in coil-reinforced sheaths, stents, stent grafts, filters, needles, wire guides, stone retrieval baskets and Vena Cava filters, among other common medical uses. The metals and alloys can be provided in any conventional form, such as flat wire, round wire, tubular or in any other form from which further processing into a desired shape can be accomplished.

The Table includes a list of significant physical properties relating to each metal or alloy. For comparison, Table 1 also includes the properties of certain well known prior art alloys, namely austenitic stainless steel, L-605, MP35N, nitinol, Elgiloy and Inconel 625.

TABLE I

| | | | | | | Young's | | | |
|---|---|---|---|---|---|---|---|---|---|
| | Metallurgical | | | | % | Modulus | Density | Xᵥ | |
| Alloy | Condition | Vendor(s) | UTS(ksi) | YS(ksi) | Elong. | (Msi) | (g/cm³) | (×10⁻⁶) | Biocompatability |
| Molybdenum/ | | | | | | | | | |
| Tungsten | | | | | | | | | |
| Alloys | | | | | | | | | |
| CP Mo | Stress relieved | Rhenium Alloys | 74.7 | 60.2 | 2-17 | 47.9 | 10.2 | <150 | Good |
| Mo—47.5Re | Annealed | Rhenium Alloys, | 171 | 123 | 22 | 52.9 | 13.5 | <110 | Good |
| | | FWM | | | | | | | |
| Mo—47.5Re | Cold worked | Rhenium Alloys, | 355-566 | | 1.5-3 | 50.8 | 13.5 | <110 | Good |
| | | FWM | | | | | | | |
| Mo—44.5Re | Annealed | Rhenium Alloys | 144 | 113 | | 52.9 | 13.5 | <110 | Good |
| W—25.5Re | | ″ | 180 | 130 | 20 | 51 | 19.7 | <110 | Good |
| Tantalum | | | | | | | | | |
| Alloys | | | | | | | | | |
| CP Tantalum | Cold worked | ″ | | | | | 16.6 | <200 | Good |
| Ta—10W | Cold worked | Robin Materials, | 141 | 132 | 1.4 | 27 | 16.8 | <175 | Good |
| | | Wah Chang, | | | | | | | |
| | | Special Metals | | | | | | | |
| Ta—8W—2Hf | Cold worked | Robin Materials, | | | | | 16.8 | <200 | Good |
| | | Wah Chang, | | | | | | | |
| | | Special Metals | | | | | | | |
| Ta—10W— | Cold worked | Robin Materials, | | | | | 16.8 | <200 | Good |
| 2.5Hf | | Wah Chang, | | | | | | | |
| | | Special Metals | | | | | | | |
| Titanium | | | | | | | | | |
| Alloys(near | | | | | | | | | |
| α) | | | | | | | | | |
| Ti—11Sn— | Duplex anneal | Allvac, Timet, | 160 | 144 | 15 | 16.5 | 4.5 | <200 | Good |
| 1Mo—2.25Al— | | Allegheny | | | | | | | |
| 5Zr—1Mo— | | Ludlum | | | | | | | |
| 0.25Si | | | | | | | | | |
| Ti—6Al—2Sn— | Duplex anneal | Allvac, Timet, | 142 | 130 | 15 | 16.5 | 4.5 | <200 | Good |
| 4Zr—2Mo | | Allegheny | | | | | | | |
| | | Ludlum | | | | | | | |
| Ti—5Al—5Sn— | Duplex anneal | Allvac, Timet, | 152 | 140 | 13 | 16.5 | 4.5 | <200 | Good |
| 2Zr—2Mo— | | Allegheny | | | | | | | |
| 0.25Si | | Ludlum | | | | | | | |

| | | | | | Young's | | | |
|---|---|---|---|---|---|---|---|---|
| | Metallurgical | | | % | Modulus | Density | Xᵥ | |
| Alloy | Condition | UTS(ksi) | YS(ksi) | Elong. | (Msi) | (g/cm³) | (×10⁻⁶) | Biocompatability |
| Ti—8Al—1Mo— | Duplex anneal | 145 | 138 | 15 | 18 | 4.4 | <200 | Good |
| 1V | | | | | | | | |
| Ti—6Al—4Zr | Duplex anneal | 148 | 132 | 6 | | 4.6 | <200 | Good |
| Titanium | | | | | | | | |
| Alloys(α + β) | | | | | | | | |
| Ti—6Al—4V | Duplex anneal | 170 | 160 | 10 | — | 4.4 | <200 | Good |
| T1—6Al—6V— | Duplex anneal | 185 | 170 | 10 | — | 4.5 | <200 | Good |
| 2.5Sn | | | | | | | | |
| Ti—6Al—2Sn— | Duplex anneal | 189 | 170 | 10 | 16.5 | 4.5 | <200 | Good |
| 4Zr—6Mo | | | | | | | | |
| Ti—7Al—4Mo | Duplex anneal | 160 | 150 | 16 | 16.5 | 4.5 | <200 | Good |
| Ti—6Al—2Sn— | Duplex anneal | 185 | 165 | 11 | 17.7 | 4.7 | <200 | Good |
| 2Zr—2Mo—2Cr | | | | | | | | |
| Titanium | | | | | | | | |
| Alloys(β) | | | | | | | | |
| IMI 834 | SHT, WQ, Age | 149 | 132 | 6 | | 4.8 | <200 | Good |
| Ti—8Mo—8V— | SHT, WQ, Age | 200 | 178 | 3 | 17.4 | 4.9 | <200 | Good |
| 2Fe—3Al | | | | | | | | |
| Ti—3Al—8V— | SHT, WQ, Age | 210 | 200 | 7 | 15.3 | 4.8 | <200 | Good |
| 6Cr—4Mo—4Zr | | | | | | | | |
| Ti—10V—2Fe— | SHT, WQ, Age | 185 | 174 | 19 | 16.2 | 4.7 | <200 | Good |
| 3Al | | | | | | | | |
| Ti—15V—3Cr— | SHT, WQ, Age | 194 | 180 | 6 | | 4.8 | <200 | Good |
| 3Al—3Sn | | | | | | | | |
| Ti—5Al—2Sn— | SHT, WQ, Age | 180 | 170 | 8 | | 4.7 | <200 | Good |
| 2Zr—4Mo—4Cr | | | | | | | | |
| Ti—13V—11Cr— | SHT, WQ, Age | 212 | 130 | 23 | 16 | 4.8 | <200 | Good |
| 3Al | | | | | | | | |
| Ti—15Mo—5Zr | SHT, WQ, Age | 187 | 133 | 14 | 14.4 | 5.1 | <200 | Good |
| Ti—11.5Mo— | SHT, WQ, Age | 180 | 174 | 6 | 15.7 | 5.1 | <200 | Good |
| 6Zr—4.5Sn | | | | | | | | |
| Ti—8Al—1Mo— | SHT, WQ, Age | 171 | 155 | 17 | 17.4 | 4.7 | <200 | Good |
| 1V | | | | | | | | |

| | | | | | | Young's | | | |
|---|---|---|---|---|---|---|---|---|---|
| | Metallurgical | | | | % | Modulus | Density | Xᵥ | |
| Alloy | Condition | Vendor | UTS(ksi) | YS(ksi) | Elong. | (Msi) | (g/cm³) | (×10⁻⁶) | Biocompatability |
| Niobium | | | | | | | | | |
| Alloys | | | | | | | | | |
| Nb—27.5Ta— | Annealed | Robin Materials, | 84.1 | 68.9 | 22 | 20.3 | 10.6 | <150 | Good |
| 11W—1Zr | Stress relieved | Wah Chang, | 109 | 106 | 11 | 20.3 | | | |
| | | Special Metals | | | | | | | |
| Nb—10W— | | Robin Materials, | 78.3 | 58 | 20 | 16 | 9 | <150 | Good |
| 2.5Zr | | Wah Chang, | | | | | | | |
| | | Special Metals | | | | | | | |
| Nb—10Hf—1Ti | Annealed | Robin Materials, | 58.7 | 50 | 26 | 12.6 | 8.9 | <150 | Good |
| | Stress relieved | Wah Chang, | 92.8 | 87.7 | 9 | 12.6 | | | |
| | Cold worked | Special Metals | 107 | 94.3 | 4.5 | 12.6 | | | |
| Nb—1Zr | Annealed | Robin Materials, | 35 | 20 | 20 | 10 | 8.6 | <150 | Good |
| | | Wah Chang, | | | | | | | |
| | | Special Metals, FWM | | | | | | | |
| Nb—1Zr | Cold worked | Robin Materials, | 230 | 185 | 3.5 | 10 | 8.6 | <150 | Good |
| | | Wah Chang, | | | | | | | |
| | | Special Metals, FWM | | | | | | | |
| CP Niobium | Cold worked | Robin Materials, | 84.8 | 30 | 5 | 14.9 | 8.6 | <150 | Good |
| | | Wah Chang, | | | | | | | |
| | | Special Metals | | | | | | | |
| Precious | | | | | | | | | |
| Metal Alloys | | | | | | | | | |
| Pt—xIr | Cold worked | Noble Met., | 70.3-200 | | | 11 | 21.5/21.7 | <230 | Good |
| (x varies 5- | Annealed | Johnson | 39.9-N/A | | | | | | |
| 30%) | | Mathey | | | | | | | |
| Pd—xAg | Annealed | Johnson | 79.8-92.8 | | | 11 | 11.7/12 | <50 | Good |
| (x varies 10- | | Mathey | | | | | | | |
| 40%) | | Deringer | | | | | | | |
| | | Ney | | | | | | | |
| Pd—xAg— | Cold worked | Johnson | 170/200 | 180 | 2.5 | 11 | 11.7/12 | <50 | Good |
| yCu(x varies | | Matthey | | | | | | | |
| from 10-40%, | | | | | | | | | |
| yvaries from | | | | | | | | | |
| 5-15%) | | | | | | | | | |
| Pd—xRe | Cold worked | Johnson | 200-230 | 190-200 | 2.5 | 11 | 12.1 | <50 | Good |
| (x varies from | | Mathey | | | | | | | |
| 1-10%) | | Deringer | | | | | | | |
| | | Ney | | | | | | | |

| | | | | | | Young's | | | |
|---|---|---|---|---|---|---|---|---|---|
| | | | | | % | Modulus | Density | Xᵥ | |
| Alloy | Comments | Vendor | UTS(ksi) | YS(ksi) | Elong. | (Msi) | (g/cm³) | (×10⁻⁶) | Biocompatability |
| Pd—xRu | Annealed | Johnson | 60 | 21 | 15 | 11 | 11.7/12 | <50 | Good |
| (x varies 1- | Stress relieved | Matthey | 75-80 | 57-62 | 6.5 | | | | |
| 10%) | Cold worked | Deringer | 90 | 82-85 | 2.2 | | | | |
| | | Ney | | | | | | | |
| Currently | | | | | | | | | |
| used alloys | | | | | | | | | |
| Austenitic SS | Annealed | Many | 70 | 30/35 | 40 | 29 | 8.0 | >3000 | Good |
| | | vendors | | | | | | | |
| Austenitic SS | Cold worked | Many | 285/315 | | | 28/29 | 8.0 | >3000 | Good |
| | | vendors | | | | | | | |
| L-605 | Annealed | Many | 135 | 70 | 35 | 35.3 | 9.2 | 1200/1600 | Good |
| | | vendors | | | | | | | |
| L-605 | Cold worked | Many | 290 | 205 | 5 | 32 | 9.2 | 1200/1600 | Good |
| | | vendors | | | | | | | |
| MP35N | Annealed | Many | 135 | 60 | 70 | 29.2 | 8.3 | 700/900 | Good |
| | | vendors | | | | | | | |
| MP35N | Cold worked | Many | 290 | 250 | 3.7 | 30 | 8.3 | 700/900 | Good |
| | | vendors | | | | | | | |
| Elgiloy | Annealed | Many | 125 | 75 | 40 | 34 | 8.4 | 1200/1600 | Good |
| | | vendors | | | | | | | |
| Elgiloy | Cold worked | Many | 300 | 270 | 4 | 34 | 8.4 | 1200/1600 | Good |
| | | vendors | | | | | | | |
| Inconnel 625 | Annealed | Many | 125 | 70 | 50 | 31 | 8.4 | | Good |
| | | vendors | | | | | | | |
| Inconnel 625 | Cold worked | Many | 295 | 220 | 4 | 31 | 8.4 | | Good |
| | | vendors | | | | | | | |
| Binary nitinol | Aged | Many | 160/200 | | | Varies | 8 | 275 | Good |
| | | vendors | | | | | | | |
| Extra stiff | Cold worked | Furukawa | | 145/200 | | | 8 | 275 | Good |
| nitinol | after aging | | | | | | | | |

As may be observed from Table 1, the magnetic susceptibility (Xᵥ) of the pure metals (CP) and the metal alloys listed is less than about 200×10⁻⁶ cgs in each case. For comparison, the magnetic susceptibility of prior art alloys is considerably higher than this. For example, the magnetic susceptibility of austenitic stainless steel (in annealed condition) is greater than 3000×10⁻⁶ cgs. The magnetic susceptibility of the alloys L-605, Elgiloy, and Inconnel 625 are generally between about 1200 and 1600×10⁻⁶ cgs, and the magnetic susceptibility of MP35N is generally between about 700 and 900×10⁻⁶ cgs. Such high magnetic susceptibility levels found in the prior art alloys may be acceptable in limited applications, however in most cases the high level of artifact created makes the alloy unsuitable for use in MR imaging. Nitinol has a better magnetic susceptibility of about 275×10⁻⁶ cgs, but suffers from low radiopacity when compared to the inventive alloys. The density levels and other physical properties of the metals and alloys used in the inventive devices will generally be at least 4 g/cm³, and therefore compare favorably with the density of the currently used alloys. Such density levels enable the devices to be readily visualized under fluoroscopy

The alloys may be prepared by processes known in the medical arts. When obtained from commercial sources, such as the sources identified in the Table, the alloys may be conventionally obtained as wire, bar, rod, foil, sheet and plate, among other forms. The metals and metal alloys referred to herein may be incorporated and/or otherwise formed by known means into medical devices, such as braided catheters, coiled catheters, stents, filters, wire guides, stent grafts, needles, among others.

Operation of medical resonance scanners has now become well known in the medical arts. The inventive medical device is compatible with conventional medical resonance scanners, and does not create an unreasonable amount of artifact. In operation, the device may be inserted into a body cavity of the patient, and transported to a point of medical diagnosis by known means. The MR scanner is then be activated by known means, and a medically useful visual readout will be obtained. If desired, x-ray fluoroscopy may also be carried out on the patient.

It is therefore intended that the foregoing detailed description be regarded as illustrative rather than limiting, and that it be understood that it is the following claims, including all equivalents, that are intended to define the spirit and scope of this invention.

1. A medical device suitable for use with MR imaging, said device comprising:
a component selected from the group consisting of a refractory metal, a precious metal, an alloy comprising a refractory metal, an alloy comprising a precious metal, an alloy comprising at least one refractory metal and at least one precious metal, and mixtures of the foregoing, said component having a magnetic susceptibility less than about 300×10⁻⁶ cgs, and having a radiopacity sufficient for visualization under fluoroscopy.

2. The device of claim 1, wherein said refractory metal comprises one or more of molybdenum, tungsten, tantalum, titanium, niobium and hafnium, and said precious metal comprises one or more of platinum, rhenium, rhodium, gold, palladium, ruthenium, silver, iridium and osmium.

3. The device of claim 2, wherein said alloy comprising a refractory metal includes at least about 50 weight percent refractory metal, said alloy comprising a precious metal includes at least about 50 weight percent precious metal, and said alloy comprising at least one refractory metal and at least one precious metal comprises at least about 50 weight percent of combined refractory metal and precious metal.

4. The device of claim 3, wherein said alloy comprising a refractory metal includes at least 70 weight percent refractory metal, said alloy comprising a precious metal includes at least 70 weight percent precious metal, and said alloy comprising at least one refractory metal and at least one precious metal comprises at least 70 weight percent of combined refractory metal and precious metal.

5. The device of claim 4, wherein said alloy comprising refractory metal includes at least 90 weight percent refractory metal, said alloy comprising precious metal includes at least 90 weight percent precious metal, and said alloy comprising at least one refractory metal and at least one precious metal comprises at least 90 weight percent of combined refractory metal and precious metal.

6. The device of claim 2, wherein said alloy is selected from the group consisting of: (a) molybdenum combined with about 5 to 50 percent rhenium; (b) tantalum combined with about 0 to 10% tungsten and/or about 0 to 3% hafnium; (c) niobium combined with about 0 to 4% zirconium and/or about 0 to 30% tantalum and/or about 0 to 15% tungsten and/or about 0 to 1% titanium; (d) tungsten combined with about 20 to 30% rhenium; (e) platinum combined with about 0 to 30% iridium; (f) palladium combined with about 0 to 30% silver and/or about 0 to 10% copper; (g) palladium combined with about 0 to 20% rhenium; and (h) palladium combined with about 0 to 20% ruthenium.

7. The device of claim 2, wherein said alloy is selected from the group consisting of: (a) molybdenum combined with 5 to 30% rhenium and 5 to 30% palladium; (b) tantalum with 5 to 40% palladium combined with about 0 to 30% silver and/or about 0 to 10% copper; and (c) niobium combined with 1 to 5% zirconium and/or 5 to 40% palladium.

8. (canceled)

9. The device of claim 2, wherein said metal or metal alloy has a magnetic susceptibility less than about 100×10⁻⁶ cgs.

10. The device of claim 2, wherein said metal or metal alloy has a density of at least about 4 g/cm³.

11. (canceled)

12. The device of claim 10, wherein said density is at least about 10 g/cm³.

13. The device of claim 1, wherein said component comprises a refractory metal selected from the group consisting of molybdenum, tungsten, tantalum, titanium, niobium and hafnium.

14. The device of claim 1, wherein said component comprises a precious metal selected from the group consisting of platinum, rhenium, rhodium, gold, palladium, ruthenium, silver, iridium and osmium.

15. The device of claim 1, wherein said component comprises an alloy comprising at least about 50 weight percent refractory metals.

16. The device of claim 1, wherein said component comprises an alloy comprising at least about 50 weight percent precious metals.

17. The device of claim 1, wherein said component comprises an alloy comprising a refractory metal and a precious metal, a combined amount of said refractory metal and said precious metal comprising at least about 50 weight percent of said device.

18. The device of claim 2, wherein said device comprises a braid or coil reinforced sheath.

19. The device of claim 2, wherein said device comprises a stent.

20. (canceled)

21. (canceled)

22. (canceled)

23. (canceled)

24. A method of diagnosing a condition in a patient under magnetic resonance imaging, comprising:
providing a medical device comprising a component selected from the group consisting of a refractory metal, a precious metal, an alloy comprising two or more refractory metals, an alloy comprising two or more precious metals, an alloy comprising at least one refractory metal and at least one precious metal, and mixtures of the foregoing, said component having a magnetic susceptibility less than about 200×10⁻⁶ cgs;
inserting said device into a cavity of the patient at a point of diagnosis;
exposing said point of diagnosis to a magnetic field;
translating a readout from said magnetic field to a visual image.

25. The method of claim 24, wherein said refractory metal comprises one or more of molybdenum, tungsten, tantalum, titanium, niobium and hafnium, and said precious metal comprises one or more of platinum, rhenium, rhodium, gold, palladium, ruthenium, silver, iridium and osmium.

26. The method of claim 25, wherein said magnetic susceptibility is less than about 100×10⁻⁶ cgs.
